(19) United States
(12) Patent Application Publication
(10) Pub. No.: US 2003/0236499 A1
(43) Pub. Date: Dec. 25, 2003

(54) Tampon applicator having a corrugated insertion tip

(76) Inventors: Glen Charles Fedyk, Township, OH (US); Ralph Edwin Neufarth, Township, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/179,808

(22) Filed: Jun. 25, 2002

(51) Int. Cl. A61M 37/00

(52) U.S. Cl. 604/150

(57) ABSTRACT

A tampon applicator is disclosed which has an insertion tip for facilitating insertion of a tampon into a woman's vagina. The tampon applicator includes a hollow first member capable of housing a tampon. The first member has a central longitudinal axis and first and second ends. An insertion tip is located on the first end of the first member and extends outwardly therefrom. The insertion tip contains an aperture that extends therethrough. The insertion tip contains a plurality of corrugations capable of expanding radially outwardly as the tampon is expelled from the first member. The tampon applicator is also disclosed in combination with a tampon.

### FIELD OF THE INVENTION

This invention relates to a tampon applicator having a corrugated tip for facilitating insertion of a tampon into a body cavity.

### BACKGROUND OF THE INVENTION

Tampons and other types of absorptive media are routinely inserted into body cavities, such as a woman's vagina, to absorb menstrual fluid, blood and other kinds of body fluid. One convenient way to position such absorbent tampons into a body cavity is through the use of an applicator. Comfortable and clean insertion of the absorbent tampon is key to repeated sale of such applicators. In addition, the applicator should be capable of inserting the absorbent tampon into the body cavity using an acceptable amount of expulsion force.

Tampon applicators are available in a variety of shapes and sizes with the two-piece telescopically assembled design being the most prevalent. In the two-piece applicator, the tampon is housed in a hollow outer tube and is expelled into a woman's vagina by an inner member that is telescopically mounted in the outer tube and acts as a plunger. Some tampon applicators utilize a hollow tube having an open insertion end through which the tampon is always exposed while other applicators utilize a completely closed or partially closed design. A thin film membrane can cover the insertion end of an applicator to completely enclose the forward end of a tampon while folds can be used to partially enclose the forward end of a tampon and protect it from contamination. Still other applicators, especially plastic applicators, have a plurality of flexible petals formed on the forward end of the outer tube which can flex radially outward to allow the tampon to be expelled. It will be appreciated that the diameter of the applicator, the material from which it is formed, the basic configuration of the applicator, the size and shape of the tampon positioned in the applicator, as well as the ease of opening the forward end of the applicator will all influence the force required to expel the tampon therefrom. The expulsion force should be kept reasonably low to permit proper functioning of the applicator.

While many have tried to design and manufacture tampon applicators having these improved qualities, there still remains a need for a tampon applicator that is more comfortable to use. Those applicators having an open forward end tend to expose the dry absorbent fibers of the tampon to the interior walls of a woman's vagina and this can cause irritation during insertion. Commercially available plastic and cardboard applicators, using a plurality of petal tips separated by slots, can sometimes pinch or cut the vaginal tissue of a woman during insertion and cause discomfort.

U.S. Pat. No. 5,782,793 issued to Nielsen et al. discloses a tampon applicator having a semi-spherically shaped pleated tip. The pleats of Nielsen are formed by doubling the material upon itself and then pressing the material into place. As can be seen in FIG. 8 of Nielsen, the pleats 50 have a thickness dimension “t” defined by three layers of material 34. The doubling of material upon itself to form the pleats leads to high expulsion forces which can cause the applicator to deform or cause the tampon to be inserted incorrectly.

Now a tampon applicator has been invented having a corrugated tip for facilitating comfortable insertion of an absorbent tampon into a woman's vagina while having a low expulsion force.

### SUMMARY OF THE INVENTION

Briefly, this invention relates to a tampon applicator having a corrugated tip for facilitating insertion of a tampon into a woman's vagina. The tampon applicator includes a first hollow member capable of housing an absorbent tampon. The first member has a central longitudinal axis and first and second ends. An insertion tip is located on the first end of the first member and extends outwardly therefrom. The insertion tip contains a central aperture having a small diameter that extends therethrough. The insertion tip contains a plurality of corrugations capable of expanding radially outward as the tampon is expelled from the first member.

The tampon applicator may include a second member telescopically mounted in the second end of the first member. The second member is adapted to expel the tampon through the insertion tip as it is pushed into the first member.

Alternatively, the first hollow member may have a string hole or opening near the insertion tip. The withdrawal string of the tampon is passed through this hole. By pulling the string through the hole the tampon can be expelled through the insertion tip without the need for a telescoping plunger. Other methods may also be used to expel the tampon from the first member without the need of a second member.

The insertion tip may be integrally formed on the first end of the first member. Alternatively, the insertion tip can be a separate member secured to the first end of the first member.

The tampon applicator is also disclosed in combination with a tampon having a shaped nose.

### BRIEF DESCRIPTION OF THE DRAWINGS

FIG. 1 is a perspective view of a two-piece, spirally wound paper tampon applicator.

FIG. 2 is a cross-sectional view of the tampon applicator shown in FIG. 1.

FIG. 3 is a left end view of the tampon applicator shown in FIG. 1 depicting the corrugations.

FIG. 3A is a left end view of a tampon applicator depicting spiral corrugations.

FIG. 4 is a cross-sectional view of the insertion tip taken along line 4-4 of FIG. 3 showing an aperture formed through the insertion tip and the aperture having a side wall aligned essentially parallel to the central longitudinal axis of the first member.

FIG. 5 is a schematic view of some corrugations taken along line 5-5 of FIG. 3 depicting the shape and thickness of the corrugations.

FIG. 6 is a cross-sectional view of an insertion tip depicting one end of the corrugations extending into the first member.

FIG. 7 is a cross-sectional view of an alternative embodiment of the insertion tip showing one end of the corrugations terminating at a point where the insertion tip integrally joins the first member.

FIG. 8 is a cross-sectional view of another embodiment of the insertion tip showing one end of the corrugations terminating at a point on the exterior surface of the insertion tip.

FIG. 9 is a perspective view of the tampon applicator showing the corrugations in an open arrangement.

FIG. 10 is a perspective view of an alternative embodiment of a hollow first member having an opening near the insertion tip.

FIG. 11 is a perspective view of an alternative embodiment of a tampon applicator insertion tip having corrugations of differing lengths.

FIG. 12 is an end view of an alternative embodiment of an insertion tip having petals.

FIGS. 13A-13B are cross-sectional views showing various tampon nose and insertion tip configurations.

### DETAILED DESCRIPTION OF THE PREFERRED EMBODIMENTS

All documents cited are, in relevant part, incorporated herein by reference; the citation of any document is not to be construed as an admission that it is prior art with respect to the present invention.

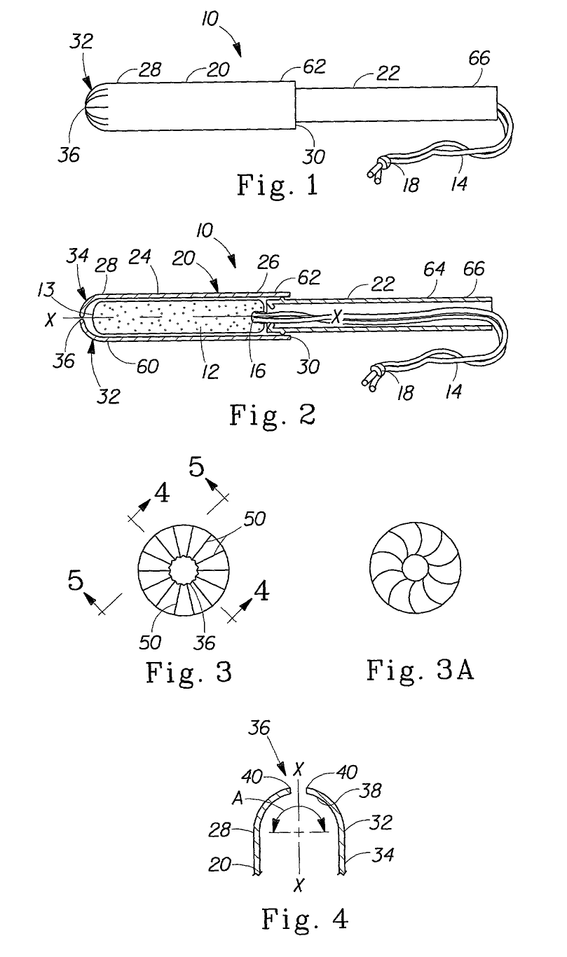

Referring to FIGS. 1-3, a tampon applicator 10 is shown which is designed to house a tampon 12 and provide a comfortable means of inserting the tampon 12 into a woman's vagina. A tampon is an absorbent member primarily designed to be worn by a woman during her menstrual period to absorb menses, blood and other body fluid. The tampon 12 can be made from natural or synthetic fibers including cellulose fibers such as cotton or rayon, or artificial fibers such as polyester, polypropylene, nylon or blends thereof. Other types of fibers may also be used, such as cellulose sponge or a sponge formed from elastomeric materials.

The tampon 12 is normally compressed into the form of a cylinder and has a forward end or nose 13. The nose 13 can be configured in any number of shapes including but not limited to blunt, rounded, semi-spherical, ellipsoidal or conical. The nose 13 shown in FIG. 2 is configured to have a semi-spherical shape. The tampon 12 commonly has a withdrawal string 14 fastened to an end thereof that serves as a means for withdrawing the soiled tampon from the woman's vagina. The withdrawal string 14 can be looped through an aperture 16 formed transversely through the tampon 12, or alternatively the string 14 can be attached to tampon 12 by known means, such as sewing. In addition, the withdrawal string 14 can have a knot 18 formed at its free end to assure that the string 14 will not separate from the tampon 12.

The tampon applicator 10 includes a first hollow member 20 and a second member 22. The first member 20 is preferably in the form of a spirally wound, convolutely wound or longitudinally seamed hollow tube which is formed from paper, paperboard, cardboard or a combination thereof. The first hollow member 20 may also be injection molded or formed from flexible plastic, such as thermoformed from plastic sheet or folded or wound from plastic film. The first hollow member 20 may also be formed from a combination of paper and plastic. The first member 20, also commonly referred to as an outer tube, is fairly rigid and has a relatively small diameter of about 10 mm to about 20 mm. The first member 20 has a wall 24 with a predetermined thickness of about 0.1 mm to about 0.7 mm. The wall 24 can be constructed from a single ply of material or be formed from two or more plies that are bonded together to form a laminate. The use of two or more plies or layers is preferred for it enables the manufacturer to use certain materials in the various layers that can enhance the performance of the tampon applicator 10. When two or more plies are utilized, all the plies can be spirally wound, convolutely wound or longitudinally seamed to form an elongated cylinder. The wall 24 can be constructed using a smooth thin ply of material on the outside or exterior surface 26 that surrounds a coarser and possibly thicker ply. When the wall 24 contains at least three plies, the middle ply can be the thicker ply and the interior and exterior plies can be smooth and/or slippery to facilitate expulsion of the tampon 12 and to facilitate insertion of the first member 20 into a woman's vagina, respectively. By sandwiching a thick, coarser ply of material between two thin, smooth plies, an inexpensive first member 20 can be provided which is very functional. The wall 24 should contain one to four plies, although more plies can be utilized if desired.

The plies forming the wall 24 can be held together by an adhesive, such as glue, or by heat, pressure, ultrasonics, etc. The adhesive can be either water-soluble or water-insoluble. A water-soluble adhesive is preferred for environmental reasons in that the wall 24 will quickly break apart when it is immersed in water.

Such immersion will occur should the first member 20 be disposed of by flushing it down a toilet. Exposure of the first member 20 to a municipal's waste treatment plant wherein soaking in water, interaction with chemicals and agitation all occur, will cause the wall 24 to break apart and evenly disperse in a relatively short period of time.

The inside diameter of the first member 20 is usually less than about 0.75 inches (about 19 mm) and preferably less than about 0.625 inches (about 16 mm). Although the exterior diameter of tampons do vary, most tampons utilized by women have an external diameter of less than about 0.75 inches (about 19 mm). However, if one desired to use this invention to administer medication to an animal, such as a farm animal or other mammal, larger size tampons 12 could be used.

It should be noted that the first member 20 can be spirally wound, convolutely wound or longitudinally seamed into a cylindrical tubular shape. Alternatively, the material can be overlapped into a tubular configuration. Spirally or convolutely winding the first member 20 into a cylindrical tube is especially advantageous when the first member 20 is formed from a laminate. The reason for this is that when a laminate is circumferentially wound into a tube and a butt seam or an overlap is formed, the butt seam or the overlap can interfere with the later formation of corrugations on the forward end thereof. A common problem with a rigid or stiff walled, tubular member having a relatively small diameter and a butt seam is that the seam has a tendency to come apart after formation if exposed to certain stress forces and/or high humidity. A problem with a tubular member having an overlap is that a small portion of the wall will be thicker than the remaining portion and this will cause problems when one tries to corrugate one end of the tube. Accordingly, the first member 20 should preferably be formed into a cylindrical configuration without the presence of a butt seam or an overlap. In the case of other tube construction methods such as fiber or plastic molding, or integral tube forming (e.g. thermoforming plastic) no seams will be present.

The first member 20 is sized and configured to house the absorbent tampon 12. As stated above, the first member 20 should have a substantially smooth exterior surface 26 that will facilitate insertion of the first member 20 into a woman's vagina. When the exterior surface 26 is smooth and/or slippery, the first member 20 will easily slide into a woman's vagina without subjecting the internal tissues of the vagina to abrasion. The first member 20 can be coated to give it a high slip characteristic. Wax, polyethylene, a combination of wax and polyethylene, cellophane and clay are representative coatings that can be applied to the first member 20 to facilitate comfortable insertion.

The first member 20 can be a straight, elongated cylindrical tube formed on a central longitudinal axis X-X. It is also possible to form the first member 20 into an arcuate shape. The arcuate or curved shape can assist in providing comfort when inserting the first member 20 into a woman's vagina. With a curved tampon applicator, it is possible to employ a curved tampon which again may be more comfortable for some women to use since the shape of the tampon may better fit the curvature of a woman's vagina.

The first member 20 has first and second spaced apart ends 28 and 30, respectively. The first member 20 can also have either a constant outer diameter or a stepped outer profile. Preferably, the first member 20 will have an essentially constant diameter over a major portion of its length. Located on the first end 28 of the first member 20 and extending outwardly therefrom is an insertion tip 32. The insertion tip 32 is designed to facilitate insertion of the first member 20 into a woman's vagina in a comfortable manner. The insertion tip 32 may be integrally formed on the first end of the first member. Alternatively, the insertion tip can be a separate member secured to the first end of the first member. The insertion tip 32 can be configured in any number of shapes including but not limited to blunt, rounded, semi-spherical or conical. The insertion tip shown in FIGS. 1 and 2 is configured to have a semi-spherical shape. The insertion tip 32 has a diameter that is approximately equal to the outside diameter of the first member 20. The insertion tip 32 has a wall 34 with a thickness that is approximately equal to the thickness of the wall 24 that forms the first member 20. However, it is possible to construct the wall 34 so that it has a thickness which is less than or greater than the thickness of the wall 24, if desired.

Referring to FIG. 4, the insertion tip 32 is shown in cross-section with the semi-spherical configuration extending outward away from the first end 28 of the first member 20. The cross-section of the semi-spherical configuration spans an arc (A) of approximately 180°. The semi-spherical configuration is formed on a diameter that is sized to be equal to or slightly smaller than the diameter of the first member 20. For example, if the outside diameter of the first member 20 is 0.64 inches (16.2 mm), the insertion tip 32 can be formed on a radius of about 0.32 inches (about 8.1 mm).

A relatively small aperture 36 is formed in the center of the semi-spherical shaped insertion tip 32 and is coaxially aligned with the longitudinal axis X-X. The aperture 36 can have a diameter of at least about 1.0 mm, preferably between about 1.0 mm to about 8.0 mm, and more preferably, between about 3.0 mm to about 4.0 mm. Another way of sizing the diameter of the aperture 36 is to make it less than about 50% of the outside diameter of the first member 20, preferably, between about 10% to about 30% of the outside diameter of the first member 20, and most preferably, less than about 20% of the outside diameter of the first member 20. It should be noted that although the aperture 36 is described as a circle, it is possible to form the aperture 36 in other shapes such as a polygon, a square, a pentagon, a hexagon, an octagon, etc. The small aperture 36 should extend through the insertion tip 32 and has a side wall 38. In addition, the edge of the side wall 38 abutting the aperture 36 can be rounded or contain a radius 40 on it's exterior surface to assure that no sharp edges are present which could pinch or cut the sensitive tissues of a woman's vagina.

Further, the edge of the side wall 38 abutting the aperture can be partially rolled inwards or folded. The purpose of the small aperture 36 in the end of the insertion tip 32 is to facilitate the subsequent unfolding of the corrugations during use, as will be described below. The aperture 36 also assures that the corrugations will symmetrically open about the longitudinal axis X-X of the first member 20. A further benefit of the aperture 36 is that it provides a visual means for the user to inspect the tampon applicator 10 and assure herself that a tampon 12 is present in the first member 20.

The configuration of a smaller aperture 36 is preferred over a larger aperture 36 as a smaller amount of the absorbent tampon 12 is exposed. Since a tampon is normally dry and consists of a plurality of absorbent fibers, it can cause abrasion against the walls of a woman's vagina as it is being inserted. By reducing the amount of surface area of the tampon 12 that is exposed to the vaginal tissue, one can decrease the level of discomfort during the insertion process. Furthermore, the small diameter of the aperture 36 also decreases the possibility of trapping or pinching vaginal tissue therein. With regard to the aperture, the exposed portion of the tampon may lie entirely below the aperture, or a portion of the tampon 12 lie within the aperture between the surrounding side wall 38, or even protrude through the aperture, though this is generally less preferred.

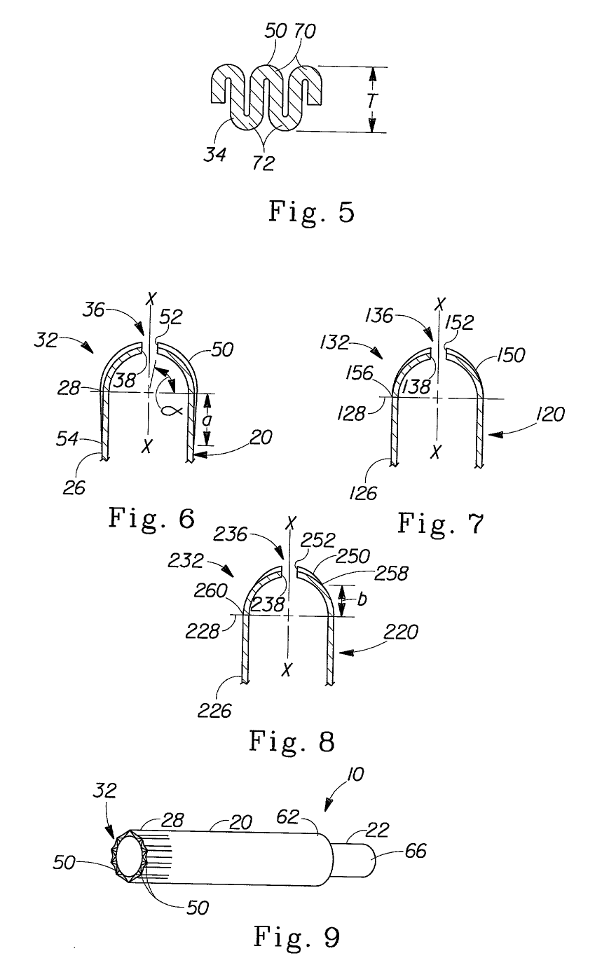

Referring to FIG. 3, the insertion tip 32 is shown having a plurality of corrugations 50 that can radially open such that the insertion tip 32 has a diameter approximately equal to the diameter of the first member 20, though it can be less than or greater than the diameter of the first member dependent upon how much material is gathered in the corrugations or how much plastic deformation may have occurred during processing. Either an even or an odd number of corrugations 50 can be present and the corrugations 50 can be equally spaced apart or they can be non-uniformly arranged. Uniformly arranged corrugations 50 are preferred but randomly arranged corrugations 50 will work. For ease of manufacturing, it is preferred that the corrugations 50 be equally spaced relative to one another. The corrugations 50 may be unequally spaced relative to one another. As seen in FIG. 5, the corrugations 50 are formed by folding the material into a series of alternating ridges 70 and troughs 72 where the transition radius at the fold between two adjoining trough or ridge walls can be tight analogous to the bottom of the capital letter “V” in Arial typefont or more gentle or curved analogous to the bottom of the capital letter “U” in Arial type-font. Further, the corrugations can be tilted such that the cross-section appears like italicized capital letters “U” and “V” in Arial type-font

Any number of corrugations 50 may be utilized. The number of corrugations 50 can vary from between 2 to about 100, preferably between about 4 to about 30. In general, the force required to open the insertion tip 32 normally increases as the number of corrugation 50 decreases. If the force required to open becomes too large, the tampon applicator 10 could bend or deform during the insertion process and this may cause discomfort. Thus, the number of corrugations 50 can be chosen to provide the desired opening force.

Referring to FIG. 5, a schematic view of a corrugation 50 is shown. The corrugation 50 is obtained by folding the paper, paperboard, or cardboard material (or alternately plastic or polymer material) so that the folding decreases the overall perimeter or circumference due to the accumulation of material with each corrugation 50 and then when each corrugation 50 is opened or unfolded it will occupy a much larger perimeter/circumference or surface area than in the previous folded state. For molded or formed plastic or fiber applicators, the corrugations could be optionally formed during the molding of the applicator. The thickness of the material forming the insertion tip 32 can be equal to or slightly less than the thickness of the first member 20, though a thickness larger than the first member 20 can also be employed. The first member 20 can have a thickness of about 0.1 mm to about 0.7 mm. The insertion tip 32 can have a thickness between about 0.1 mm to about 0.7 mm. In the folded condition, the corrugation 50 has a thickness, indicated by the letter “t”. The thickness “t” of each corrugation 50 in the folded condition will be greater than the thickness of the material from which the insertion tip 32 is constructed.

The material in the insertion tip 32 region can be optionally subjected to other process steps either before or after folding the material to obtain the corrugations. An example would be mechanical scoring or embossing of the material to soften the tip by working the fibers in a paper tube first member 20.

Referring to FIGS. 6-8, three different embodiments of a corrugation are depicted. In FIG. 6, the corrugation 50 is depicted as having a first end 52 that coincides with the side wall 38 of the aperture 36. In other words, the first end 52 of the corrugation 50 forms a portion of the arc of the aperture 36. The corrugation 50 also has a second end 54 that coincides with a point located on the exterior surface 26 of the first member 20. This point is spaced a distance “a” from the location where the insertion tip 32 is integrally joined to the first member 20. By forming the corrugation 50 with this particular length, one can control the amount of force needed to open the insertion tip 32 and push the tampon 12 therethrough.

In FIG. 6, the semi-spherical tip 32 spans a radial arc, identified as angle alpha (α), which extends from the first end 52 to the point where the semi-spherical shaped tip 32 is integrally joined to the first member 20. The angle alpha (α) is generally between about 60° to about 90°, preferably between about 75° to about 90°, and most preferably, greater than 80°. The angle alpha (α) would be 90° if the aperture 36 were not present. The size of the aperture 36 will partially determine the exact angle of the insertion tip 32. The angle alpha (α) should be as close to 90° as possible without completely enclosing the nose 13 of the tampon 12.

In FIG. 7, an alternative embodiment of an insertion tip 132 is depicted wherein a corrugation 150 is shown having a first end 152 which coincides with the side wall 138 of the aperture 136. In other words, the first end 152 of the corrugation 150 forms a portion of the arc of the aperture 136. The corrugation 150 also has a second end 156 that coincides with the point where the insertion tip 132 is integrally joined to the first end 128 of the first member 120. By forming the corrugation 150 with this particular length, one can control the amount of force needed to open the insertion tip 132 and push the tampon 12 therethrough. Although the force required to open the corrugations 150 may be slightly greater than the force required with the design shown in FIG. 6, the force is still within acceptable limits.

In FIG. 8, a third embodiment of an insertion tip 232 is depicted wherein a corrugation 250 is shown having a first end 252 which coincides with the side wall 238 of the aperture 236. In other words, the first end 252 of the corrugation 250 forms a portion of the arc of the aperture 236. The corrugation 250 also has a second end 258 that coincides with a point located on an exterior surface 260 of the insertion tip 232. This point is spaced a distance “b” from the location where the insertion tip 232 is integrally joined to the first end 228 of the first member 220. By forming the corrugation 250 with this particular length, one can control the amount of force needed to open the insertion tip 232 and push the tampon 12 therethrough. Although the force required to open the corrugations 250 may be greater than the force required with the designs shown in FIGS. 6 and 7, the force is still within acceptable limits.

The corrugations for a given insertion tip may be formed to have essentially identical lengths. Alternatively, the corrugations may be of differing lengths. For example, the lengths of the corrugations may be selected to alternate between a first length dimension and a second length dimension which are different from one another. The differing lengths may be random or arranged in a pattern. The number of differing length dimensions may be more two or more.

Referring now to FIG. 11, a tampon applicator 410 is shown having a first hollow member 420 and a second member 422. The first member 420 has an insertion tip 432. The insertion tip 432 includes a plurality of corrugations 450 of differing length dimensions. The result is a higher concentration of corrugations 450 near the aperture 436.

The corrugation shape and orientation can vary. For example the corrugations may form lines that parallel or lie within a given radial plane such as in FIG. 3. Alternatively, the corrugations may form curves or spirals that cross several radial planes. An example of corrugations forming spirals crossing several radial planes is shown in FIG. 3A.

The corrugations may also include cuts to improve the fold or unfolding of the insertion tip. The cuts can comprise scores on either the interior or exterior surface, small perforations or even longer cuts, which could create sidewall slits or even discrete petals. The cuts can be oriented and located in any direction or region of the insertion tip as desired.

Regarding the length of the corrugations, they could extend below the tip of the first member 20 toward the second end of the first member 20, even to the point of corrugating the entire first member 20 and just close the insertion tip at the first end.

Referring now to FIG. 12, the insertion tip 532 may be separated into a plurality of triangular segments or petals 580. The petals 580 converge around a central aperture 536. The sides 582 of the petals 580 are separated from one another by narrow spaces 584. Each petal 580 includes a plurality of corrugations 550. Any number of petals may be selected.

It should be noted that both the length and diameter of commercially available tampons do vary and therefore the tampon applicator 10 can be manufactured in a variety of accommodating sizes. Tampons can vary in length from about 1 to about 3 inches (about 25.4 mm to about 76.2 mm) but preferably are about 2 inches (about 50.8 mm) in length. The tampon diameter will also vary from about 0.25 inches to about 0.75 inches (about 6.4 mm to about 19.0 mm). In addition, the material from which the tampon 12 is constructed, the smoothness of the internal surface of the first member 20, the shape of the second member 22, etc. all contribute to establish a needed expulsion force to open and expel the tampon 12. This force should range from between about 250 grams to about 1,500 grams, preferably less than about 1,200 grams, and most preferably, less than about 1,000 grams. A lower force value is preferred for it assures that the tampon applicator 10 will be less susceptible to being bent or deformed as the tampon 12 is expelled. An unintentionally bent applicator could cause the tampon to be inserted incorrectly. A lower force value also makes the tampon applicator 10 easier to use.

Referring again to FIGS. 1 and 2, the first member 20 can have a fingergrip area 62 located approximate the second end 30. The fingergrip area 62 can be integrally formed from the material from which the first member 20 is constructed or it can be a separate member that is secured in place by an adhesive or some other type of attachment mechanism. The fingergrip area 62 functions to provide a means for the user to grip the first member 20 and hold it between her thumb and finger. The user can then position another finger on the free end of the second member 22 and orient the first member 20 relative to her vagina while she pushes the second member 22 into the first member 20.

As stated above, the tampon applicator 10 includes a second member 22, also commonly referred to as an inner tube. The second member 22, like the first member 20, can be a spirally wound, a convolutely wound or a longitudinally seamed hollow tube constructed from paper, paperboard, cardboard, or a combination thereof. The second member 22 can also be formed into a cylindrical tube by overlapping the material upon itself. The second member 22 may also be injection molded or thermoformed from flexible plastic. The second member 22 may also be formed from a combination of paper and plastic. The second member 22 can be constructed of the same material as the first member 20 or it can be made out of a different material. Furthermore, the second member 22 could be constructed as a laminate having two or more plies which are then spirally wound, convolutely wound or longitudinally seamed into a cylindrical tube. Either a wound tube or a longitudinally seamed tube is preferred because the finished tube will have a wall 64 with a constant thickness. However, some manufacturers may prefer to construct the second member 22 as a solid stick or use some other unique shape. It is also possible to form a fingergrip area 66 on the outer end of the second member 22 to provide a surface onto which the user's finger can rest.

Referring to FIG. 9, the second member 22 functions by being telescopically movable relative to the first member 20. As the second member 22 is pushed into the first member 20, the tampon 12 is forced forward against the corrugations 50. The contact by the tampon nose 13 causes the corrugations 50 to radially open to a diameter that is sufficient to allow the tampon 12 to be expelled from the first member 20. The open arrangement of the corrugations 50 is shown in FIG. 9 after the tampon 12 has been expelled. With the tampon 12 properly positioned in the woman's vaginal cavity, the tampon applicator 10 is withdrawn and properly discarded.

In a preferred configuration in order to reduce the expulsion force, the tampon nose 13 should have minimal contact with the corrugations 50. By maintaining contact between the tampon nose 13 and the corrugations 50 to a minimum the expulsion force is distributed over a smaller surface area allowing the corrugations to be unfolded with a lower total force exerted by the user. One way to have minimal contact is to configure the tampon nose 13 and the insertion tip 32 of different geometries. A tampon applicator 10 having a semi-spherically shaped insertion tip 32 works well in combination with a tampon having an ellipsoidally shaped nose. This mismatch in geometry ensures minimal contact between the tampon nose 13 and the corrugations 50 of the insertion tip 32. Other ways of minimizing the contact between the corrugations 50 and the tampon nose 32 may also be used. Also, for certain situations, a particularly advantageous principle for selecting the nose shape is such that the minimal contact area with the corrugations is located in the region surrounding the aperture. In this way, the applied force gains leverage in opening the corrugations.

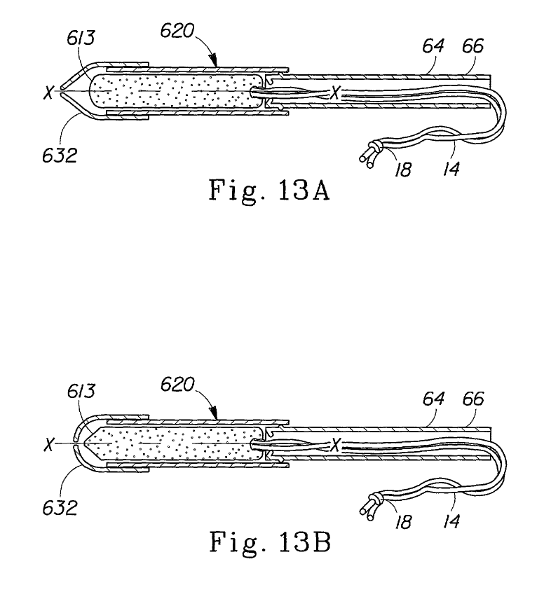

Referring now to FIGS. 13A-13B, there is shown several different tampon nose and insertion tip configurations. FIG. 13A shows a tampon nose 613 having an ellipsoidal configuration and an insertion tip 632 having a conical configuration. FIG. 13B shows a tampon nose 613 having a conical configuration and an insertion tip having a 632 having an ellipsoidal configuration. The insertion tips 632 shown in FIGS. 13A and 13B are separate members attached to the first member 620.

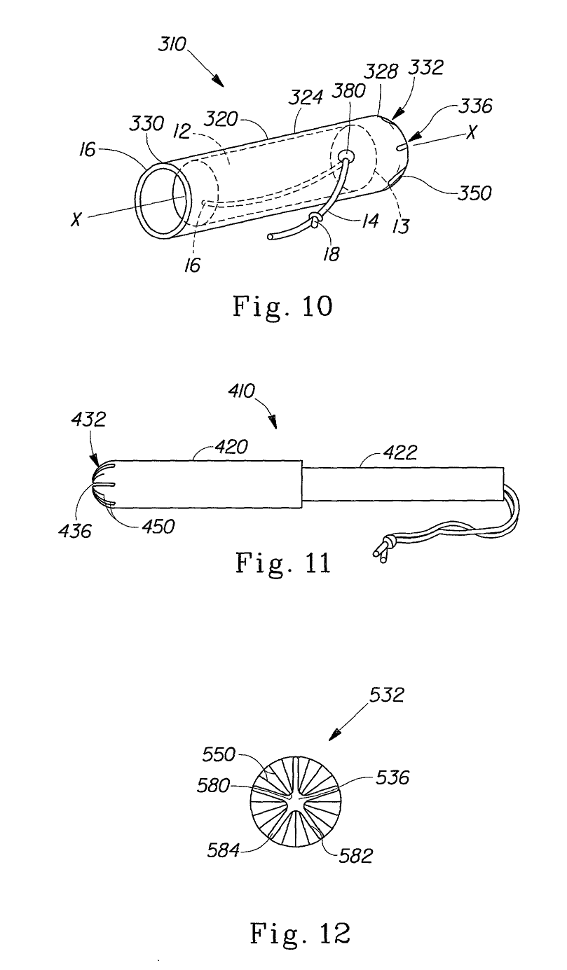

Referring now to FIG. 10, a tampon applicator 310 is shown which is designed to house a tampon 12 and provide a comfortable means of inserting the tampon 12 into a woman's vagina. The tampon applicator 310 includes a first hollow member 320 having a wall 324 and first and second spaced apart ends 328 and 330, respectively. Integrally formed on the first end 328 of the first member 320 and extending outwardly therefrom is an insertion tip 332. A relatively small aperture 336 is formed in the center of the insertion tip 332 and is coaxially aligned with the longitudinal axis X-X. The insertion tip 332 has a plurality of corrugations 350 which can radially open. A string hole 380 is located in the side wall 324 adjacent the first end 328.

The tampon 12 has a nose 13 at one end and a withdrawal string 14 fastened to the opposite end. The withdrawal string 14 can be looped through an aperture 16 formed transversely through the tampon 12. In addition, the withdrawal string 14 can have a knot 18 formed at its free end to assure that the string 14 will not separate from the tampon 12. The withdrawal string 14 extends through the string hole 380 adjacent first end 328 and insertion tip 332. By pulling on the string 14 through the string hole 380 the tampon 12 is expelled through the insertion tip 332.

While the invention has been described in conjunction with several specific embodiments, it is to be understood that many alternatives, modifications and variations will be apparent to those skilled in the art in light of the foregoing description. Accordingly, this invention is intended to embrace all such alternatives, modifications and variations that fall within the spirit and scope of the appended claims.

We claim:

1. A tampon applicator comprising:
a) a hollow first member capable of housing a tampon, said first member having a central longitudinal axis, a first end and a second end; and
b) an insertion tip located on said first end of said first member and extending outwardly therefrom, said insertion tip having an aperture extending therethrough, said insertion tip including a plurality of corrugations capable of expanding outward as said tampon is expelled from said first member.

2. The tampon applicator of claim 1 further comprising a second member telescopically mounted in said second end of said first member, said second member adapted to expel said tampon through said insertion tip as said second member is pushed into said first member.

3. The tampon applicator of claim 1 wherein said corrugations cross several radial planes.

4. The tampon applicator of claim 1 wherein said corrugations are arranged to form a configuration selected from the group consisting of semi-spherical, conical and ellipsoidal.

5. The tampon applicator of claim 1 wherein said corrugations are of are varying length dimensions.

6. The tampon applicator of claim 1 wherein said first member is selected from the group consisting of spirally wound, longitudinally seamed and convolutely wound.

7. The tampon applicator of claim 1 wherein said insertion tip is integrally formed on said first end of said first member.

8. The tampon applicator of claim 1 wherein said first member is constructed from plastic.

9. The tampon applicator of claim 1 wherein said first member is constructed from at least one ply of paper.

10. The tampon applicator of claim 1 wherein said insertion tip comprises a plurality of cuts.

11. In combination, a tampon applicator and a tampon having a shaped nose, said combination comprising:
a) a tampon;
b) a hollow first member capable of housing said tampon, said first member having a central longitudinal axis, a first end and a second end; and
c) an insertion tip located on said first end of said first member and extending outwardly therefrom, said insertion tip having an aperture extending therethrough, said insertion tip including a plurality of corrugations capable of expanding outward as said tampon is expelled from said first member.

12. The combination of claim 11 further comprising a second member telescopically mounted in said second end of said first member, said second member adapted to expel said tampon through said insertion tip as said second member is pushed into said first member.

13. The combination of claim 11 wherein said corrugations cross several radial planes.

14. The combination of claim 11 wherein said corrugations are arranged to form a configuration selected from the group consisting of semi-spherical, conical and ellipsoidal.

15. The combination of claim 11 wherein said corrugations are of varying length dimensions.

16. The combination of claim 11 wherein said first member is selected from the group consisting of spirally wound, longitudinally seamed and convolutely wound.

17. The combination of claim 11 wherein said tampon nose has a configuration selected from the group consisting of conical, ellipsoidal and semi-spherical.

18. The combination of claim 11 wherein said tampon nose has a configuration different than the configuration of said insertion tip.

19. The combination of claim 11 wherein said first member is constructed from at least one ply of paper.

20. The combination of claim 11 wherein said insertion tip is integrally formed on said first end of said first member.
